(19) United States
(12) Patent Application Publication
(10) Pub. No.: US 2016/0263173 A1
(43) Pub. Date: Sep. 15, 2016

(54) PAIN RELIEF FORMULATION AND METHOD OF TREATMENT

(71) Applicant: ATP INSTITUTE PTY LTD, Loganholme, Queensland (AU)

(72) Inventor: Matthew Legge, Loganholme (AU)

(21) Appl. No.: 15/032,200

(22) PCT Filed: Oct. 28, 2014
(86) PCT No.: PCT/AU2014/050314

§ 371 Date: Apr. 26, 2016

(30) Foreign Application Priority Data
Oct. 28, 2013 (AU) 2013904159

(51) Int. Cl. A61K 36/61; A61K 31/165; A61K 36/38; A61K 47/46; A61K 36/67; A61K 47/12; A61K 45/06; A61K 9/00; A61K 31/045; A61K 31/618

(52) U.S. Cl. CPC A61K 36/61; A61K 31/045; A61K 31/165; A61K 36/38; A61K 31/618; A61K 36/67; A61K 47/12; A61K 45/06; A61K 9/0014; A61K 47/46; A61M 35/003

(57) ABSTRACT

A topically administrable pain relief formulation comprises a Kunzea ambigua extract or one or more components or derivatives thereof; Menthol; Capsaicin; and a Hypericum perforatum extract or one or more components or derivatives thereof. The formulation may further comprise methyl salicylate, black pepper and/or an anti-inflammatory agent. The formulation may be used to treat or prevent pain such as arthritis neck pain, shoulder pain, back pain, preoperative and/or postoperative pain and pain associated with minor or traumatic injuries or other diseases or conditions.

### TECHNICAL FIELD

THIS INVENTION relates to topical pain relief formulations and methods of treatment. The formulations are useful for a variety of pain relieving applications such as for example the treatment of joint pain.

### BACKGROUND TO THE INVENTION

The development of safer and more effective methods for reducing or eliminating pain using topical formulations is an ongoing process. Over time, a variety of topical formulations have been developed. These include lotions, gels and ointments containing any number of non-steroidal anti-inflammatory drugs (NSAIDS) such as aspirin.

However, many current topical pain agents are not entirely satisfactory and can have side effects. For example, opioids can cause tolerance, dependence, constipation, respiratory depression and sedation, NSAIDS have gastrointestinal side effects, can cause renal damage, can increase bleeding time, and are not effective in the treatment of severe pain and non-selective sodium channel blockers, can cause central nervous system (CNS) side effects, cardiovascular side effects and corneal damage after use.

While many of the currently available pain relieving topical formulations reduce pain to some degree, there is, nonetheless, a continued interest in identifying new formulations which provide longer lasting pain relief in a short period of time without undesirable side effects.

Accordingly, there is continued interest in the development of new topical pain relief agents.

### SUMMARY OF THE INVENTION

The present invention is directed to formulations and methods for treating pain in a subject.

In a broad form, the invention relates to a pain relief formulation, such as for reducing, alleviating and/or preventing pain such as for example pain associated with arthritis, arthralgia, rheuinatism, infection, myalgia, muscle damage and neuralgia.

In a first aspect the invention provides a topically administrable pain relief formulation comprising:
(i) a Kunzea ambigua extract or one or more components or derivatives thereof;
(ii) Menthol;
(iii) Capsaicin; and
(iv) a Hypericum perforatum extract or one or more components or derivatives thereof.

In one embodiment, the pain relief formulation comprises from about 0.01% to about 75% of Kunzea ambigua extract or one or more components or derivatives thereof.

Preferably, the pain relief formulation comprises about 5% Kunzea ambigua extract or one or more components or derivatives thereof or about 25 to 75% Kunzea ambigua extract or one or more components or derivatives thereof. Suitably, the Kunzea ambigua extract is Kunzea ambigua oil.

In one embodiment, the pain relief formulation comprises from about 0.01% to about 10% of menthol. Preferably, the pain relief formulation comprises about 5% menthol.

In one embodiment, the pain relief formulation comprises from about 0.01 to about 10% of capsaicin. Preferably, the pain relief formulation comprises about 0.035% capsaicin or about 2.5% capsaicin.

In one embodiment, the pain relief formulation comprises from about 0.01% to about 20% of Hypericum perforatum extract or one or more components or derivatives thereof. Preferably, the pain relief formulation comprises about 0.25% Hyperictun perforatum extract or one or more components or derivatives thereof or about 10% Hypericum perforatum extract or one or more components or derivatives thereof. Suitably, the Hypericum perforatum extract is Hypericum perforatum essential oil or infused Oil.

The topical formulation may further comprise methyl salicylate.

In one embodiment, the pain relief formulation comprises from about 0% to about 20% of methyl salicylate. Preferably, the pain relief formulation comprises about 5% methyl salicylate or about 10% methyl salicylate.

In one embodiment, the topical administrable formulation further comprises at least one pharmaceutically acceptable carrier, diluent and/or excipient. Preferably, at least one other pharmaceutically acceptable carrier, diluent and/or excipient may include one or more of a solubiliser, emollient, moisturiser, thickener, skin conditioner, preservative and/or stabiliser as would be understood by a person of skill in the art.

In one embodiment, the pharmaceutically acceptable carrier is an oil. Preferably, the pharmaceutically acceptable carrier is selected from the group consisting of: grape seed (Vitis vinifera) oil, Olive (Olea europaea) oil, Jojoba (Simmondsia chinenis seed) oil, Rosehip (Rosa canina, Rosa Mosqueta or Rosa Rubiginosa) oil.

The formulation may further comprise at least one additional terpene. Preferably, the terpene is black pepper (Piper nigrum) extract or one or more components or derivatives thereof. More preferably, the black pepper extract or one or more components or derivatives thereof is black pepper essential oil.

In one embodiment, the pain relief formulation comprises from about 0.01% to about 10% of black pepper extract or one or more components or derivatives thereof. Preferably, the pain relief formulation comprises about 0.25% black pepper extract or one or more components or derivatives thereof or about 2.5% black pepper extract or one or more components or derivatives thereof.

In another embodiment, the formulation further comprises a penetration enhancer. Preferably the penetration enhancer is oleic acid.

In one embodiment, the formulation further comprises 0.01% to 99% horse chestnut extract or one or more components or derivatives thereof. Suitably, the formulation comprises horse chestnut extract or one or more components or derivatives thereof at a concentration of 1%.

In one embodiment, the formulation further comprises an anti-inflammatory agent. Suitably, at least one anti-inflammatory agent is selected from the following non-limiting examples: steroidal anti-inflammatories, non-steroidal anti-inflammatories, herb and health supplements, dietary anti-inflammatories, anti-inflammatory oils and any other known sources of anti-inflammatories.

In one embodiment, the topical formulation is provided as an oil, mousse, gel, cream, lotion, foam, ointment, liniment, liquid, aerosol and the like.

Preferably, the topical formulation is an oil, cream or lotion.

In one embodiment, the present invention provides a formulation in the form of a pain relief topical application. The formulation comprises Kunzea arnbigua extract or one or more components or derivatives thereof at 0.01-7.5%, menthol at 0.01-10%. capsaicin at 0.01-10%, Hypericum perforatum extract or one or more components or derivatives thereof at 0.01-20%; a terpene such as black pepper essential oil at 0.01-10%; at least one carrier such as grape seed oil at 0.01%-99%, olive oil at 0.01%-99%, jojoba oil at 0.01%-99%, rosehip oil 0.01%-99%; and optionally methyl salicylate at 0-20%.

In another embodiment, the formulation comprises Kunzea ambigua extract or one or more components or derivatives thereof at 0.01-75%. menthol at 0.01-10%, capsaicin at 0.01-10%, Hypericum perforatum extract or one or more components or derivatives thereof at 0.01-20%; a terpene such as black pepper essential oil at 0.01-10%; a carrier such as grape seed oil, at 0.01-99%, olive oil at 0.01-99%, jojoba oil at 0.01-99%, rosehip oil at 0.01-99%; horse chestnut 1:1 fluid extract at 0.01-75% 1%; and optionally methyl salicylate at 0-20%.

In another embodiment, the formulation comprises Kunz,ea anibigua extract or one or more components or derivatives thereof at 0.01-75%, menthol at 0.01-10%, capsaicin at 0.01-10%. Hypericum perforatum extract or one or more components or derivatives thereof at 0.01-20%; a terpene such as black pepper essential oil at 0.01-10%; a carrier such as grape seed oil at 0.01-99%; and optionally methyl salicylate at 0-20%.

In a second aspect, the invention provides a method of producing the topical formulation according to the first aspect, including the step of micronizing the capsaicin before combining the micronized capsaicin with other ingredients to produce the topical pain relief formulation.

In one embodiment, micronization of the dry ingredients is achieved through milling, bashing, macerating and/or grinding.

In a third aspect, the invention provides a method of treating or preventing pain in a subject, said method including topically administering to said subject an effective amount of a topical pain relief formulation according to the first aspect or produced according to the method of the second aspect. to treat the subject for pain or to prevent pain in the subject.

In a fourth aspect, the invention provides a topically administrable pain relief formulation according to the first aspect or produced according to the method of the second aspect for use in the therapeutic and/or prophylactic treatment of pain.

In one embodiment, the pain relief formulation is topically administered to a subject at a location proximal to said pain.

In one embodiment, the subject being treated is suffering from pain selected from the group consisting of: arthritis pain, neck pain, shoulder pain, back pain, surgical pain, preoperative and/or postoperative pain, bone injury pain, muscle pain: pain associated with muscle tension, fatigue, curvature of the spine, minor and major spinal disc compression, pinched nerves, strained or sprained muscles, nervous tension and delayed onset muscle soreness (DOMS); pain associated with traumatic injuries, hematomas, myositis, lower back syndromes, spinal stenosis, joint pain, hone pain and hone fractures caused by cancer; pain associated with osteoprotic fractures of the lumbar spine and other sites; traumatic bone fractures; pain associated with pre-surgical and post-surgical orthopedic procedures; pain caused by a slipped disc, musculo-skeletal pain, joint dislocations, herniated intervetebral disc, prolapsed intervetebral disc, ruptured disc, whiplash injuries, fibromyositis, intercostal rib pain, muscle tear, tendonitis, bursitis, meniscal tears, tendon tears, and hone spurs; pain associated with muscle spasms, pain caused by sports related injuries, hematomas, bruises, sprains, muscle spasms, partial tendon tears, tendonitis. bursitis, myositis, traumatic arthritis and post-insertion of joint dislocation; neuralgia; and pain caused by diseases, disorders or conditions.

In one embodiment, the subject is a mammal. Preferably the subject is a human.

Alternatively, the subject is a non-human mammal, non-limiting examples of which include a horse, dog cat, rabbit and the like.

In a fifth aspect, the invention provides a kit comprising the topically administrable pain relief formulation according to the first aspect or produced according to the method of the second aspect, an applicator device and instructions for using said formulation to provide pain relief to a subject in need thereof.

In one embodiment, the applicator device is a roll-on device.

Throughout this specification, unless otherwise indicated, “comprise”, “comprises” and “comprising” are used inclusively rather than exclusively, so that a stated integer or group of integers may include one or more other non-stated integers or groups of integers.

As used in this specification the indefinite articles “a” and “an” may refer to one entity or a plurality of entities and are not to be read or understood as being limited to a single entity.

### DETAILED DESCRIPTION

The present invention relates to formulations and methods for treating pain in a subject, examples of which include arthritic pain, muscle pain or neuralgia.

The present inventors have created an improved formulation which, when topically applied to the skin can aid in the treatment of pain. The present invention provides a formulation and method for treating pain, such as for example arthritic pain.

### Pain Relief Topical Formulations

In a first aspect, the invention relates to a topical formulation for the treatment of pain, comprising:
(i) Kunzea ambigua extract or one or more components or derivatives thereof;
(ii) Menthol;
(iii) Capsaicin; and
(iv) Hypericum perforatum extract or one or more components or derivatives thereof.

As used herein, the term “extract” refers to a composition or preparation comprising one or more active components, compounds or substances obtained, isolated or extracted from a particular source. The active components, compounds or substances in the extract may be in a more concentrated or enriched form compared to the source. In particular, the extract may be obtainable from a plant or any portion thereof, including for example the native Australian plant Kunzea ambigua (Myrtaceae family) and the perennial herb Hypericum perforatum or St. John's Wort.

The term “derivative” refers to a modified form of a particular compound or substance. The derivative may be a modified form of a compound or substance that is a component of, present in, or obtainable from, for example a Kunzea ambigua extract and/or a Hypericum perforatum extract. Other examples include derivatives of menthol and/or capsaicin and Horse chestnut extract. Typically, the derivative is a chemically modified or related form of the particular compound or substance.

In one embodiment, the formulation comprises Kunzea ambigua extract or one or more components or derivatives thereof at a concentration from about 0.01, 1, 2, 3, 4, 5, 6, 7, 8, 9, 10, 11, 12, 13, 14, 15. 16, 17, 18, 19, 20, 21, 22, 23, 24, 25, 30, 35, 40, 45 to 50% by weight, from the Australian native plant Kunzea ambigua. Preferably, the Kunzea ambigua extract is Kunzea ambigua oil.

Suitably, the pain relief formulation may be provided in a dilute or concentrated form. Preferably, the dilute formulation comprises about 5% Kunzea ambigua extract and the concentrated formulation comprises about 25 to 75% Kunzea ambigua extract. Preferably, the Kunzea ambigua extract is Kunzea ambigua oil.

Suitably, Kunzea ambigua oil comprises the following components: Alpha-pinene 52%; 1,8 cineole 12%; Alpha-terpineol 2%; Bicyclogermacrene 4.4%; Globtdol 7.6%; Viridiflorol 6.8%; <1% of linalool; teminene-4-ol; citronellol; allo-aromadendrene; caryophyllene; ledol and spatholenol.

Kunzea ambigua oil is a safe, non-toxic essential oil which is well tolerated on the skin even with undiluted use.

Alternative names and/or synonyms for Kunzea ambigua may include for example Leptospermum ambiguum, Kunzea bracteolate, Kunzea calida, Kunzea ericoides (Kanuka) (syn. K. pedunculata), Kunzea graniticola, Kunzea opposite, Kunzea acicularis, Kunzea acuminate. Kunzea affmis, Kunzea baxteri, Kunzea bracteolate, Kunzea cambagei, Kunzea capitata. Kunzea oricifolia, Kunzea eriocalyx, Kunzea flavescens. Kunzea glabrescens, Kunzea jucunda, Kunzea micrantha, Kunzea mictomera, Kunzea montana, Kunzea muelleri, Kunzea newbyi, Kunzea obovata, Kunzea parvifolia Kunzea pauciflora, Kunzea pomifera, Kunzea preissiana, Kunzea pulchella, Kunzea recurva, Kunzea rupestris, Kunzea spicata, Kunzea strigosa, Kunzea villiceps or other related species in the myrtle family Myrtaceae.

In one embodiment, the formulation comprises menthol at a concentration of about 0.01, 0.02, 0.03, 0.04, 0,05, 0.06, 0.07, 0.08, 0.09, 0.10, 0.20, 0.30, 0.40, 0.50, 0.75, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 to 10%. Preferably, menthol is at a concentration of about 5%.

In one embodiment, the formulation comprises capsaicin at a concentration of about 0.01, 0.015. 0.02, 0.025, 0.03, 0.035, 0,04, 0.045. 0.05, 0.06, 0.07, 0.08, 0.09, 0.10, 0.20, 0.30, 0.40, 0.50, 0.75, 1.0, 1.5, 2.0, 2.5, 3.0, 3.5, 4.0, 4.5. 5.0, 5.5, 6.0, 6.5, 7.0, 7,5, 8,0, 8.5, 9.0, 9.5 to 10%. Preferably, capsaicin is at a concentration of about 0.035% in the dilute formulation and about 2.5% in the concentrated formulation.

In one embodiment, the formulation comprises Hypericum perforatum extract or one or more components or derivatives thereof at a concentration of about 0.01, 0.02, 0.03, 0.04, 0.05, 0,06, 0,07, 0.08, 0.09, 0.10, 0.20,0.25, 0.30, 0.35, 0.40, 0,45, 0.50, 0.55, 0.60, 0.65, 0.70, 0.75, 0.80, 0.85, 0.90. 0.95, 1.0, 1.5, 2.0, 2.5, 3.0, 3.5, 4.0, 4.5, 5.0, 55, 6.0, 6.5, 7.0, 7.5, 8.0. 8.5, 9.0, 9.5, 10, 11.12, 13, 14, 15, 16, 17, 18. 19 to 20%. Preferably, Hypericum perforatum extract or one or more components or derivatives thereof is at a concentration of about 0.25% in the dilute formulation and about 10% in the concentrated formulation. Preferably, the Hypericum perforatum extract is Hypericum perforatum essential oil.

In one embodiment, the topical formulation further comprises methyl salicylate.

In one embodiment the formulation comprises methyl salicylate at a concentration of about 0.01, 0.02, 0.03, 0.04, 0.05, 0.06, 0.07. 0.08, 0.09, 0.10, 0.20,0.25, 0.30, 0.35, 0.40. 0.45, 0.50, 0.55, 0.60. 0.65, 0.70, 0.75, 0.80, 0.85. 0.90, 0,95, 1,0, 1.5, 2.0, 2,5, 3.0, 3.5, 4.0, 4,5, 5.0, 5.5, 6.0. 6.5, 7.0, 7.5, 8.0, 8.5, 9.0, 9.5. 10. 11, 12, 13, 14. 15, 16, 17, 18, 19 to 20%. Preferably, methyl salicylate is at a concentration of about 5% in the dilute formulation and about. 10% in the concentrated formulation.

The topical formulation of the invention may further comprise a pharmaceutically acceptable carrier, diluent or excipient. These include without limitation an effective amount of a moisturising, solubilising and/or substantive ingredient. A useful reference describing; pharmaceutically acceptable carriers, diluents and excipients is Remington's Pharmaceutical Sciences (Mack Publishing Co. NJ USA, 1991).

In one embodiment, the carrier is an oil, examples of which include without limitation Grape (Vitis vinifera) seed oil, Olive (Olea europaea) Jojoba (Simmondsia chinenis seed) oil, Rosehip (Rosa canina, Rosa mosqueta or Rosa rubiginosa) oil, Rapeseed oil, canola oil, avocado oil, peanut oil. corn oil, truffle oil, coconut oil. sesame oil. palm oil. flaxseed oil, hazelnut oil, pumpkin seed oil, almond oil, apricot kernel oil, Moroccan oil, tamanu oil, clove oil, basil oil, nutmeg oil, rosemary oil or lavender oil.

Preferably, the carrier is selected from the group consisting of grape seed (Vitis vinifera) oil, Olive (Olea europaea) oil, Jojoba (Simmondsia chinenis seed) oil and Rosehip (Rosa canina, Rosa mosqueta or Rosa rubiginosa) oil.

In one embodiment, grape seed oil (Vitis vinifera) comprises fatty acids Rookie, linolenic, oleic, palmitic and stearic.

In one embodiment, the formulation comprises grape seed oil at a concentration from about 0.01 to about 99%.

In one embodiment, Olive (Olea europaea) oil comprises fatty acid components-Palmitic, Palmitoleic, Stearic, Oleic, Linoleic, Linolenic, Arachidic, Gadoleic; Hydrocarbons, examples of which include squalene and β-carotene; Tocaphemls including for example vitamin E; and fatty alcohols and waxes.

Suitably, the formulation comprises olive oil at a concentration from about 0.01 to about 99%. Preferably. the olive oil is at a concentration of about 15%.

In one embodiment. Jojoba (Siinmondsia chinenis seed) oil comprises fatty acid components Palmitic, Palmitoleic, Stearie, Oleic, Linoleic, Linolenic, Arachidic, Eicosenoic, Behenic, Erucic, Lignoceric; and Iodine.

In one embodiment, the formulation comprises jojoba oil at a concentration from about 0.01 to about 99%. Preferably, the jojoba oil is at a concentration of about 5%.

Jojoba oil is highly stable, does not oxidize, volatilize, or become rancid after standing for long periods of time. Repeated heating to temperatures above 285° C. for 4 days and exposure to high pressure does not alter its properties.

In another embodiment, rosehip oil comprises fatty acids oleic acid (20%), linoleic acid (41%) and linolenic acid (39%), trans retinoic acid; red and yellow pigments, including for example carotenoids beta-carotene, lycopene, rubixanthin, gazaniaxanthin, beta-cryptoxanthin, and zeaxanthin together with other minor carotenoids (violaxanthin, antheraxanthin, and gamma-carotene); and essential fats. Rosehip enhances transdermal absorption of actives.

In one embodiment, the formulation comprises rosehip oil at a concentration from about 0.025 to about 99%. Preferably, the rosehip oil is at a concentration of about 1%.

In addition to the above carrier, the formulation may also comprise Terpenes (Aqil, M et al., 2007, Drug Discovery Today, Vol 12, Issues 23-24, Pages 1061-1067; and Williams, A. C., and Barry, B. W., 1991, Pharmaceutical Research, vol. 8. Issue 1, Pages 17-24).

In one cribodiment. terpenes include without Limitation the following examples; Hemiterpenes, Monoterpenes, Sesquiterpenes, Diterpenes. Sesterterpenes, Triterpenes, Tetraterpenes, Polyterpenes. Alternatives may include Geraniol, Citronellol, Camphor, Pinenes (α and β)—Pine genera. Borneol, Rutaceae; Myrtaceae; Umbel lifereae; Labiatae; Compositae; Pinaceae Bergamot, Citronella, Laurel, Vetiver, Ginger, Sandalwood, Cinnamon, Nutmeg. cannabis sativa. Mentha×piperita, Mentha canadenis, Mentha spicata L. (M. viridis Linn.) and Mentha×cardiac, Mentha arvensis, oils are derived from Eucalyptus polybractea. Eucalyptus smithii. Eucalyptus australiana and Eucalyptus globulus.

Preferably, the terpenes in the formulation include black pepper extract or one or more components or derivatives thereof and menthol. More preferably, black pepper extract is black pepper essential oil.

Suitably, black pepper essential oil comprises Limonene, Pinene. Myrcene, Phellandrene. Beta-caryophyllene. Beta-bisabolene, Sabinene, Linalol. Pinocarveol, Alpha Terpineol, Camphene and Alpha Terpenene.

In one embodiment, the formulation comprises black pepper essential oil at a concentration from about 0.01 to about 10%. Preferably, the black pepper essential oil is at a concentration of about 0.25%.

Terpenes are included in the list of Generally Recognized As Safe (GRAS) substances and have low irritancy potential. Their mechanism of percutaneous permeation enhancement involves increasing the solubility of drugs in skin lipids, disruption of lipid/protein organization and/or extraction of skin micro constituents that are responsible for maintenance of barrier status.

Terpenes are compounds naturally found in many essential oils including those from black pepper. Terpenes can enhance the permeation of both hydrophilic and lipophilic drugs. Black pepper essential oil induces a warming sensation when applied to the skin due to local dilation of microcirculation to the skin, which is capable of enhancing percutaneous absorption of the active ingredients.

In one embodiment, an effective amount of a penetration enhancer may be incorporated into the pain relief formulation to aid penetration of the active ingredients. Suitably, penetration enhancers include without limitation: Oleic acid, 2 N-nonyl-1,3-dioxolanes, N-acetyle prolinate esters (such as pentyl- and octyl-N-acetyle prolinate), alkyldiloxanes (e.g., 1-Alkyl-3-b-D gincopyranosyl-1,1,3.3-tetramediyl disiloxanes), transcarbam (such as 5-(dodecyloxycarbonyl) pentylammonium-5-(dodecyloxycarbonyl) pentylcarbamate), iminosulfurane (like N-hexyl,N-benzoyl-S,S-dimethyliniino-sulfuranes), capsaicin derivatives (e.g., Nortivamide), cinnamene compounds (such as cinnamic acid, cinnamaldehyde etc). terpenes, penetration enhancers have been discussed including fatty acids, terpenes, fatty alcohol, pyrrolidone, sulfoxides, laurocapram, surface active agents, amides, amines, lecithin, polyols, quaternary ammonium compounds, silicones, alkanoates and cardamom seed.

Preferably, the penetration enhancer comprises oleic acid (Louk. A et al., 1995, Journal of Controlled Release. Vol, 37. Issue 3, Pages 299-306).

Oleic acid may be sourced from any of the following oils without limitation, including olive oil, grape seed oil, pecan oil, canola oil, peanut oil, macadamia oil, sunflower oil, sea buckthorn oil, and sesame oil and poppy seed oil. Preferably the oleic acid is sourced from olive oil and/or grape seed oil.

In one embodiment, the formulation further comprises horse chestnut extract or one or more components or derivatives thereof. Suitably, the formulation comprises horse chestnut extract or one or more components or derivatives thereof at a concentration of 1%.

In one embodiment, the formulation further comprises an anti-inflammatory. Suitably, at least one anti-inflammatory is selected from the following non-limiting examples: steroidal anti-inflarnmatories, non-steroidal anti-inflammatories, herb and health supplements, dietary anti-inflammatories, anti-inflammatory oils and any other known sources of anti-inflammatories.

Steroidal anti-inflammatories may include for example Amcinonide, Betarnethosone diproprionate. Clobetasol, Clocortolone, Dexamethasone, Diflorasone, Dutasteride, Flumethasone Pivalate, Flunisolide, Huocinolone Acetonide, Fluocinonide, Fluorometholone Fluticasone propionate, Fluticasone propionate, Fluticasone propionate. Flurandrenolide and Hydroflumethiazide.

Non-steroidal anti-inflammatories may include for example aspirin. ibuprofen, naproxen, ketoprofen, diclofenac, celecoxib and meloxicam.

Anti-inflammatory herb and health supplements may include for example devil's claw (Harpagophytum procumbens), hyssop, hops (Humulus opulus), licorice (Glycyrrhiza glabra), galangal, ginger (Zingiber officinale), turmeric (Curcuma longa), nutmeg, chickweed, comfrey ginsengs, danshen, sage, rosemary. thyme, coriander, fenugreek, golden seal, saffron, calendula, bilberry, elderberry. Arnica montana (containing helenalin), rehmannia, willow hark (containing salicylic acid) and glycosaminoglycans (glucosamine, chondroitin, and MSM).

Dietary anti-inflammatories may include for example pomegranate (Punica granatum), green tea (Camellia sinensis), cat's claw (Uncaria tometosa and Uncaria guianensis). Indian olibaum (Boswelia serrata), flavonoids (quercetia, rutin, hesperidin, luteolin), Greenlipped mussel, acai, olive leaf. West African sorghum, red yeast rice, cacao, pawpaw/papaya (papain) and pineapple bromelain (Ananas comosus).

Anti-inflammatory oils may include for example Omega 3, 6, 7 and 9, conjugated linoleic acid (CLA) which may be obtained from various sources.

Examples of other anti-inflammatories, include for example three-amino acid peptide phenylalanine-glutamine-glycine (FEG) and its D-isomeric form. Carmabichromene (a cannabinoid), Honokiol. Black seed extract or a component or derivative thereof (Nigella sativa) and hypeiforin (St. John's wort chief constituent).

The pain relief formulation preferably has the consistency of an oil. In some embodiments, the pain relief formulation has a semi-solid consistency of a mousse, gel, cream or lotion for ease of storage and application.

In one embodiment, an effective amount of a thickener may be incorporated within the formulation to obtain the desired viscosity and consistency of the product for example, as use as a cream, lotion or ointment. Suitable thickening agents include acrylate polymers and co-polymers among the classes of polyacrylates, polytnethacrylate, polymethyltnethacrylates, polyacrylamides and their cross-linked derivatives, cellulose derivatives such as methyl cellulose, hydroxyethyl cellulose, hydroxypropylmethyl cellulose, and hydroxypropyl cellulose, sorbitol (glucitol), naturally derived gums such as xanthan gum, acacia gum, carob gum; alginate derivatives, pectin, carbomer, trolamine. glycerine and polysorbate.

The thickener is incorporated in an amount suitable to obtain the desired thickening effect.

Additionally, other ingredients such as but not limited to antioxidants, pH modifiers, perfumes, preservatives, and colours may be included within the formulation.

In one embodiment, the pain relief formulation may be delivered using the following non limiting examples: carbomer gel, serum, spray, bath oils, massage oils, patches, nanopatches, nanodelivery systems, compresses, poultices, tape, bandages, sports strapping tape, dose delivery devices, slow release devices, epidermal injection, subcutaneous injection, dropper, clothing, dressings, gauze and/or intra-articular injection.

### Method of Production

In a second aspect, the invention provides a method of producing the topical formulation according to the first aspect, including the step of micronizing the capsaicin before combining the micronized capsaicin with the remaining ingredients to produce the topical pain relief formulation.

In one embodiment, mieronization of the dry ingredients is achieved through the following non-limiting examples: milling, bashing, maceration and/or grinding, mechanical impact mills (e.g., hammer mills and pin mills), fluid energy mills (e.g., spiral jet mills, pancake mills, loop jet mills or fluidized bed jet mills), RESS (Rapid Expansion of Supercritical Solutions) process, the SAS (Supercritical. Anti-Solvent) method and the PGSS method (Particles from Gas Saturated Solutions) and Nanoparticalization.

The micronized capsaicin may be combined with the remaining ingredients in any order to form the pain relief formulation. Preferably, the micronized capsaicin is combined with Kunzea ambigua extract or one or more components or derivatives thereof, Menthol, Capsaicin and Hypericum perforatum extract or one or more components or derivatives thereof, prior to the addition of any optional ingredients.

### Methods of Treatment and use of the Topical Formulation

The topical formulation of the invention finds use in methods of treating a subject for pain, wherein the subject is known to be suffering from pain and the topical formulation is employed to treat the pain.

In an aspect, the invention provides a method of treating or preventing pain in a subject, said method comprising topically administering to said subject a therapeutically effective amount of the topical pain relief formulation disclosed herein and/or produced according to the method disclosed herein to treat the subject for pain or to prevent pain in the subject.

In an aspect, the invention provides the topically administrable pain relief formulation disclosed herein and/or produced according to the method disclosed herein for the therapeutic and/or prophylactic treatment of pain.

The terms “administration” or “administered” describe the introduction of the topical pain relief formulation, to a subject's skin.

The term “therapeutically effective amount” describes a quantity of the formulation of the first aspect to achieve a desired effect in a subject being treated with the formulation. For example, this can be the amount of the formulation necessary to reduce, alleviate and/or prevent pain in a subject In some embodiments, a “therapeutically effective amount” is sufficient to reduce or eliminate pain. In other embodiments, a “therapeutically effective amount” is an amount sufficient to achieve a reduction or decrease in pain.

Suitably, a therapeutically effective amount of the pain relief formulation is an amount sufficient to induce the desired result without causing a substantial cytotoxic effect in the subject. The effective amount of the formulation of the first aspect or produced according to the method of the second aspect, useful for eliminating, reducing, alleviating and/or preventing pain will be dependent on the subject being treated, the type and severity of any associated disease, disorder and/or condition, and the manner of administration of the pain relief formulation.

By “reducing”, as in reducing pain in a subject, is meant a lessening or shortening of pain or of the length of time a subject experiences pain. Such reducing need not be absolute to be beneficial to the subject. By “alleviating”, as in alleviating pain in a subject, is meant a reduction in the severity or seriousness of the pain. Such alleviating need not be absolute to be beneficial to the subject. Reduction and/or alleviation of pain in a subject can be determined using any methods or standards known to the ordinarily skilled artisan, including both qualitative and quantitative methods and standards.

In some embodiments, the subject is experiencing pain.

In other embodiments, a “prophylactic” treatment is administered to a subject who does not exhibit signs of pain or exhibits only early signs for the purpose of decreasing the risk of developing pain.

In practicing the methods of the invention, the topical formulation may be administered to any topical site on a subject. Topical sites of interest include without limitation: arms, leg, torso, head, etc. The surface area that is covered by the topical formulation following application must be sufficient to provide for the desired amount of formulation to alleviate pain.

In one embodiment, the period of time that the topical pain relief formulation is maintained at the site of application is about 48 hours. In a further embodiment, the time that the topical pain relief formulation is maintained at the site of application is about 24 hours.

Suitably, the period of time during which the formulation is maintained at the application site is at least about 1, 2, 3. 4, 5. 10, 15, 20, 30, 45. 50 minutes, 1, 1.5, 2, 3, 3.5, 4. 5, 6, 7. 8, 9, 10, 12, 15, 20, 24, 48 or 72 hours.

In one embodiment, a given dosage of the topical pain relief formulation may be applied as a single application or a plurality of applications over a given time period, e.g., for as long as the subject feels pain, where the dosing schedule is administered over a given time period, examples of which include hourly, daily, weekly, biweekly or monthly dosing schedules.

In one embodiment, the topical formulation is applied at a pain site of the subject, wherein the phrase “pain site” is hereinafter defined as referring to the location of pain as perceived by a subject.

In one embodiment, the pain site may be present at multiple locations on a subject. The pain site or application site to which the topical formulation is applied will be sufficiently proximal to the pain felt by the subject, so that upon application of the formulation, the components of the formulation can readily reach the pain site and actively work to treat such pain.

Suitably, the topical pain relief formulation is generally applied to the pain site for a period of time sufficient for the desired amount of pain relief to be achieved.

If pain recurs following removal or non-use of the topical pain relief formulation, further topical formulation may be applied. The process may be repeated as necessary and when desired by the subject to achieve pain relief.

In some embodiments, the patient may experience relief from the pain either immediately or shortly after application. Suitably, the patient will experience at least some relief from the pain about 1 to 60 seconds; 1, 2, 3, 4, 5, 6. 7, 8, 9, 10, 15, 20, 30, 40, 50, 60 minutes or 1, 1.5, 2, 2.5, 3,4. 5, 6, 7, 8, 9, 10, 15, 20, 25 hours or days following application of the topical formulation.

The amount of topical pain relief formulation applied will usually he sufficient to cover the area of skin overlying the pain site so that the subject experiences pain relief. For solutions, liquids, gels, lotions, creams, ointments and the like, the topical formulation may be applied to the pain site and a covering optionally applied thereto. For example, such covering may include patches, bandages, plasters and dressings. An appropriate sized covering may be placed over the applied topical pain relief formulation. Conveniently, the topical formulation may be provided in a unit dosage dispenser, such as for example a pump bottle, spray, dropper or roll-on device examples of which are well known in the art.

Upon application of the topical formulation, the components of the formulation penetrate the surface of the skin and the subject experiences pain relief. Suitably, the subject experiences at least a partial reduction in the intensity of pain. Preferably, the subject experiences almost total pain relief and in some cases may experience a complete cessation of pain. Accordingly, application of the topical formulation in accordance with the methods of the invention results in treatment of the subject suffering from pain.

The topical formulation of the invention is available to a plurality of subjects. The term “subject” is used in its broadest sense. In a preferred embodiment, the subject is a mammal. More preferably, the subject is a human. Non-limiting examples of mammals include humans, dogs, cats, horses, cows, sheep, goats and pigs. Preferably, a subject includes any human or non-human mammal, including for example, a primate, cow, horse, pig, sheep, goat, dog, cat, or rodent, capable of being treated for pain relief.

By “treatment” is meant at least an amelioration of the pain experienced by a subject, where amelioration is used in a broad sense to refer to at least a reduction in the magnitude of a parameter, e.g, pain rating, associated with the condition, disorder or disease being treated. As such, treatment also includes situations where the pain is completely inhibited, e,g. prevented from happening, or stopped, e.g. terminated, such that the host no longer suffers from the pain. The beneficial effect of the pain relief formulation can be determined using any methods or standards known to a person of skill in the art.

The topical pain relief formulation of the invention may be used to treat pain associated with many conditions by topically applying the formulation to the pain site as described in the aforementioned embodiments. Specifically, the topical formulation may be used to treat pain, including, but not limited to arthritis, neck pain, shoulder pain, back pain, surgical pain, preoperative and postoperative pain and bone injury pain.

In some embodiments, the topical formulation may also be used to treat pain associated with osteoarthritis, auto-immune diseases such as rheumatoid arthritis and psoriatic arthritis, gout, pseudo gout, ankylosing spondylitis, juvenile arthritis, systemic lupus erythematosus, arthritis and neuralgia associated with an infection, scleroderma and fibromyalgia.

In other embodiments, the topical formulation may be used to treat muscle pain, pain associated with muscle tension, fatigue, curvature of the spine, minor and major spinal disc compression, pinched nerves, strained or sprained muscles, nervous tension and delayed onset muscle soreness (DOMS). Moreover, the topical formulation may be used to treat pain associated with traumatic injuries, hematomas, myositis, lower back syndromes, spinal stenosis, joint pain bone pain and bone fractures caused by metastic cancer, such as breast or lung. Suitably, the topical formulation may also be used to treat muscle, bone and joint pain generally associated with cancer. The present formulation may be used to treat pain associated with osteoporotic fractures of the lumbar spine and other sites, and traumatic bone fractures, including pelvic fractures. With respect to joint pain, the topical formulations may be used to decrease overall joint stiffness and increase joint mobility.

In one embodiment, the topical formulation may also be used to treat pain associated with pre-surgical and post-surgical orthopedic procedures. For example, the present formulation may be applied to treat such pain before or after arthroscopy, especially in the shoulders or knees. In addition, the present formulations may be used for treating pain associated with post-surgical orthopedic recovery, such as tendon, muscle and bone repair, as well as joint replacement, including hip or knee replacement. For example, bone fractures require the use of plates, screws or other attachment means to hold the bones together. Placement of these devices requires surgery, and the post-surgical pain resulting there from can be treated with the topical formulations of the invention.

In one embodiment, the topical formulations may be used to treat pain caused by a slipped disc, musculo-skeletal pain, joint dislocations, herniated intervertebral disc, prolapsed intervertebral disc (including lumbar and cervical), ruptured disc, whiplash injuries, fibromyositis, intercostal rib pain, muscle tear, tendonitis, bursitis, meniscal tears, tendon tears, and bone spurs. The topical formulation may also be used to treat pain associated with muscle spasms.

The formulation may be used to treat pain caused by sports related injuries, including but not limited to, hematomas, bruises, sprains, muscle spasms, partial tendon tears, tendonitis, bursitis, myositis, traumatic arthritis and post-insertion of joint dislocation.

The topical formulation may also be used in combination with local or other injections of an anesthetic, such as lidocane.

In addition, the topical pain relief formulation may be used in combination with oral analgesics or anti-inflammatories (e.g., NSAIDS) to alleviate pain.

The topical formulation of the invention may also be used in combination with heat treatment devices including, but not limited to, hot packs such as heating pads or hot towels to provide an enhanced and/or additive pain relief effect.

Further, the present pain relief formulation may be used in combination with morphine-like agents, such as codeine, opiates, oxy-cotcontin, Percocet, Demorol and Vicadin.

The present pain relief formulation can also be used in combination with acupuncture therapy. The topical formulation herein may provide an enhanced and/or additive relief effect when used in combination with acupuncture.

### Kits

According to a fifth aspect, the invention resides in a kit comprising the pain relief formulation according to the first aspect or produced according to the method of the second aspect, an applicator device and instructions for using said formulation to provide pain relief to a subject in need thereof.

The applicator device can be any device that ensures correct and targeted delivery of the pain relief formulation of the first aspect. Examples of appropriate devices include applicators which attach to a single- or multi-dose container of the formulation, tubes, roll-on devices or droppers.

Preferably, the applicator device is a roll-on device.

The kit may also include instructions for how to use the formulation, where the instructions typically include information about how to apply the formulation, dosing schedules etc. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging) etc. In other embodiments, the instructions are present as an electronic storage data file.

### EXAMPLES

The topical formulation of this invention may be provided as oil, mousse, gels, creams, lotions, foams, liquids, aerosols and the like.

The following examples and accompanying tables provide embodiments of the pain relief formulations of the invention, methods for preparing same and methods of administration to a subject.

### Example 1

A topical pain relief formulation was prepared comprising 5% Kunzea ambigua oil, 5% menthol, 0.035% capsaicin and 0.25% Hypericum perforatum essential oil as per the following formulation (Table I) to provide a dilute formulation suitable to be used for example in a roll-on device.

The formulation may be used in the treatment of pain relating to for example arthritis, neuralgia, myalgia, gout, ciguatera toxin, shingles (herpes zoster), varicella zoster virus, chickenpox, HSV-1 and HSV-2 lesions.

The required quantity of capsaicin was micronized to form a powder. In a separate container, the active ingredients, including Hypericum perforatum essential oil. Kunzea ambigua oil and menthol were combined and the micronized capsaicin was incorporated using a geometric dilution to form an active blend.

In a further separate contained the pharmaceutically acceptable carrier oils, including black pepper essential oil, grape seed oil, olive oil, jojoba oil and rosehip oil were combined and subsequently added to the active blend to form a topical pain relief formulation.

Optionally, Methyl salicylate may be added to the pan relief formulation.

The formulation may then be transferred to a suitable application device such as for example a roll-on device, which allows for an easy, no mess application.

### Example 2

A topical pain relief formulation was prepared comprising 5% Kunzea ambigua oil, 5% menthol, 0.035% capsaicin and 0.25% Hypericum perforatum essential oil as per the following formulation (Table 2) to provide a dilute formulation suitable to he used for example in a roll on device.

The formulation may be used in the treatment of pain relating to for example varicose veins.

The required quantity of capsaicin was micronized to form a powder. In a separate container, the active ingredients, including Hypericum perforatum essential oil, Kunzea ambigua oil and menthol were combined and the micronized capsaicin was incorporated using a geometric dilution to form an active blend.

In a further separate contained the carrier oils, including black pepper essential oil, grape seed oil, olive oil, jojoba oil and rosehip oil were combined and subsequently added to the active blend together with about 1% horse chestnut 1:1 fluid extract to form a topical formulation.

Optionally. Methyl salicylate may be added to the pan relief formulation.

The topical pain relief formulation may then be transferred to a suitable application device such as for example a roll-on device, which allows for an easy, no mess application.

### Example 3

A topical pain relief formulation was prepared comprising 25 to 50% Kunzea ambigua oil, 5% menthol, 2.5% capsaicin and 10% Hypericum perforatum essential oil as per the following formulation (Table 3) to provide a concentrated formulation suitable to be used for example in a dropper device for dispensing small amounts of concentrated topical pain relief formulation.

The formulation may be used in the treatment of pain relating to for example arthritis, neuralgia, myalgia, gout, ciguatera toxin, shingles (herpes zoster), varicella zoster virus, chicken pox. HSV-1 and HSV-2 lesions.

The required quantity of capsaicin was micronized to form a powder. In a separate container, the active ingredients, including Hypericum perforatum essential oil. Kunzea ambigua oil and menthol were combined and the micronized capsaicin was incorporated using a geometric dilution to form an active blend.

In a further separate contained the carrier oils, including grape seed oil, black pepper essential oil, olive oil, jojoba oil and rosehip oil were combined and subsequently added to the active blend to form a topical pain relief formulation.

Optionally, Methyl salicylate may be added to the pan relief formulation.

The formulation may then be transferred to a suitable application device such as for example a dropper device, which allows for the concentrated application of active ingredient.

Formulations according to the present invention provide many advantages over the prior art. They may be applied over a large or small area of the skin surface of a subject and effectively treat pain relating to a number of diseases and disorders or conditions as hereinbefore described, without any side effects.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

In particular, it is noted that the concentrations of ingredients may be readily varied by a person of skill in the art. It will also be appreciated that. concentrations expressed in the range x-y % include all ranges within the stated range.

All patent and scientific literature referred to herein is incorporated herein by reference.

TABLE 1

| | |
|---|---|
| | Roll-on device |
| Application | Dilute formulation |
| | Arthritis, neuralgia, myalgia, gout, Ciguatera toxin, |
| | Shingles (herpes zoster), Varicella zoster virus, |
| | chickenpox, HSV-1 and HSV-2 lesions |
| | Carrier oil base with penetration enhancers |
| Ingredients | Grapeseed oil 1-99% |
| | Olive oil 15% (1 to 99%) |
| | Jojoba oil 5% (1 to 99%) |
| | Black pepper essential oil 0.25% (0.01 to 10%) |
| | Rosehip Oil 1% (0.025 to 99%) |
| | Active ingredients |
| | Kunzea ambigua oil 5% (0.01% to 75%) |
| | Menthol 5% (0.01 to 10%) |
| | Optional Methyl Salicylate 5% (0 to 20%) |
| | Capsaicin 0.035% (0.01 to 10%) |
| | Hypericum perforatum essential oil 0.25% (0.01 to |
| | 20%) |
| Functional | Acute and chronic pain relief for neuralgia, myalgia, |
| Properties of each | articular, soft tissue, ligament pain and inflammatory, |
| formulation | toxic, infectious, trauma, acute or chronic injury |
| | triggers. |

TABLE 2

| | |
|---|---|
| | Roll-on device |
| Application | Dilute formulation |
| | Varicose veins |
| | Carrier oil base with penetration enhancers |
| Ingredients | Grapeseed oil 1-99% |
| | Olive oil 15% (1 to 99%) |
| | Jojoba oil 5% (1 to 99% |
| | Black pepper essential oil 0.25% (0.01 to 10%) |
| | Rosehip Oil 1% (0.025 to 99%) |
| | Active ingredients |
| | Kunzea ambigua oil 5% (0.01% to 75%) |
| | Menthol 5% (0.01 to 10%) |
| | Optional Methyl Salicylate 5% (0 to 20%) |
| | Capsaicin 0.035% (0.01 to 10%) |
| | Hypericum perforatum essential oil 0.25% |
| | (0.01 to 20%) |
| Additional Ingredients | Horse chestnut 1:1 fluid extract 1% |
| Functional Properties of | Anti-inflammatory and antioxidant for vascular |
| each formulation | integrity. |

TABLE 3

| | |
|---|---|
| | Concentrated topical drops for pain relief |
| Application | Arthritis, neuralgia, myalgia, gout, Ciguatera toxin, |
| | Shingles (herpes zoster), Varicella zoster virus, |
| | chickenpox, HSV-1 and HSV-2 lesions |
| | Carrier oil base with penetration enhancers |
| Ingredients | Grapeseed oil 20% (1-99%) |
| | Black pepper essential oil 2.5% (0.01 to 10%) |
| | Active ingredients |
| | Kunzea ambigua oil 25-50% (0.01% to 75%) |
| | Menthol 5% (0.01 to 10%) |
| | Optional Methyl Salicylate 10% (0 to 20%) |
| | Capsaicin 2.5% (0.01 to 10%) |
| | Hypericum perforatum essential oil 10% (0.01 to 20%) |
| Functional | Acute and chronic pain relief for neuralgia, myalgia, |
| Properties of each | articular, soft tissue, ligament pain and inflammatory, |
| formulation | toxic, infectious, trauma, acute or chronic injury |
| | triggers. |

1. A topically administrable pain relief formulation comprising:
a Kunzea ambigua extract or one or more components or derivatives thereof;
(ii) Menthol;
(iii) Capsaicin; and
(iv) a Hypericum perforatum extract or one or more components or derivatives thereof.

2. The pain relief formulation of claim 1, wherein the formulation comprises from about 0.01% to about 75% of Kunzea ambigua extract or one or more components or derivatives thereof.

3. The pain relief formulation of claim 1, wherein the formulation comprises about 5% to about 25 to 50% of Kunzea ambigua extract or one or more components or derivatives thereof.

4. The pain relief formulation of claim 1, wherein the Kunzea ambigua extract or one or more components or derivatives thereof is Kunzea ambigua oil.

5. The pain relief formulation of claim 1, wherein the formulation comprises from about 0.01% to about 10% of menthol.

6. The pain relief formulation of claim 1, wherein the formulation comprises about 5% menthol.

7. The pain relief formulation of claim 1, wherein the formulation comprises from about 0.01% to about 10% of capsaicin.

8. The pain relief formulation of claim 1, wherein the formulation comprises about 0.035% or about 2.5% capsaicin.

9. The pain relief formulation of claim 1, wherein the formulation comprises from about 0.01% to about 20% of Hypericum perforatum extract or one or more components or derivatives thereof.

10. The pain relief formulation of claim 1, wherein the formulation comprises about 0.25% or about 10% Hypericum perforatum extract or one or more components or derivatives thereof.

11. The pain relief formulation of claim 1, wherein the Hypericum perforatum extract or one or more components or derivatives thereof is Hypericum perforatum essential oil.

12. The pain relief formulation of claim 1, wherein the formulation further comprises methyl salicylate.

13. The pain relief formulation of claim 12, wherein the formulation comprises from about 0% to about 20% methyl salicylate.

14. The pain relief formulation of claim 12, wherein the formulation comprises about 5% or about 10% methyl salicylate.

15. The pain relief formulation of claim 1, further comprising at least one pharmaceutically acceptable carrier, diluent and/or excipient.

16. The pain relief formulation of claim 15, wherein the pharmaceutically acceptable carrier is an oil.

17. The pain relief formulation of claim 1, further comprising at least one additional terpene.

18. The pain relief formulation of claim 17, wherein the terpene is black pepper extract or one or more components or derivatives thereof.

19. The pain relief formulation of claim 18, wherein the black pepper extract or one or more components or derivatives thereof is black pepper essential oil.

20. The pain relief formulation of claim 1, further comprising a penetration enhancer.

21. The pain relief formulation of claim 20, wherein the penetration enhancer is oleic acid.

22. The pain relief formulation claim 1, further comprising an anti-inflammatory agent.

23. The pain relief formulation of claim 1, wherein the formulation is an oil, mousse, gel, cream, lotion, lubricant, foam, liquid or aerosol.

24. A method of producing the topically administrable pain relief formulation according to claim 1, including the step of micronizing the capsaicin before combining the micronized capsaicin with the other ingredients to thereby produce the topical pain relief formulation.

25. A method of treating or preventing pain in a subject, said method including:
topically administering to said subject an effective amount of a topical pain relief formulation of claim 1, to treat the subject for pain or to prevent pain in the subject.

26. A topically administrable pain relief formulation according to claim 1, for the therapeutic and/or prophylactic treatment or prevention of pain.

27. The method of claim 25, wherein the pain is selected from the group consisting of: arthritis pain, neck pain, shoulder pain, back pain, surgical pain, preoperative and/or postoperative pain, bone injury pain, muscle pain; pain associated with muscle tension, fatigue, curvature of the spine, minor and major spinal disc compression, pinched nerves, strained or sprained muscles, nervous tension and delayed onset muscle soreness (DOMS); pain associated with traumatic injuries, hematomas, myositis, lower back syndromes, spinal stenosis, joint pain, bone pain and bone fractures caused by cancer; pain associated with osteoprotic fractures of the lumbar spine and other sites; traumatic bone fractures; pain associated with pre-surgical and post-surgical orthopedic procedures; pain caused by a slipped disc, musculo-skeletal pain, joint dislocations, herniated intervertebral disc, prolapsed intervetebral disc, ruptured disc, whiplash injuries, fibromyositis, intercostal rib pain, muscle tear, tendonitis, bursitis, meniscal tears, tendon tears, and bone spurs; pain associated with muscle spasms, pain caused by sports related injuries, hematomas, bruises, sprains, muscle spasms, partial tendon tears, tendonitis, bursitis, myositis, traumatic arthritis and post-insertion of joint dislocation; neuralgia; and pain caused by other diseases, disorders or conditions.

28. The method of claim 25, wherein the subject is a human.

29. A kit comprising the pain relief formulation according to claim 1, an applicator device and instructions for using said formulation to provide pain relief to a subject in need thereof.

30. The kit according to claim 29, wherein the applicator device is a roll-on device.
